(19) United States
(12) Patent Application Publication
(10) Pub. No.: US 2020/0375963 A1
(43) Pub. Date: Dec. 3, 2020

(54) TARGETING MITOCHONDRIAL COMPLEX II TO REDUCE EFFECTS OF CHRONIC HYPOXIA

(71) Applicant: Health Research, Inc., Buffalo, NY (US)

(72) Inventor: Bora E. BAYSAL, Orchard Park, NY (US)

(21) Appl. No.: 16/485,390

(22) PCT Filed: Feb. 9, 2018
(86) PCT No.: PCT/US2018/017630

§ 371 Date: Aug. 12, 2019

Related U.S. Application Data
(60) Provisional application No. 62/457,557, filed on Feb. 10, 2017.

(51) Int. Cl. A61K 31/4412; A61P 11/00; A61K 33/00

(52) U.S. Cl. CPC A61K 31/4412; A61K 33/00; A61P 11/00

(57) ABSTRACT

Provided are methods for treatment of chronic systemic hypoxia. The method comprises administration of an inhibitor of mitochondrial complex II (MTCII). An example of an MTCII inhibitor is Atpenin 5.

### CROSS-REFERENCE TO RELATED APPLICATIONS

This application claims priority to U.S. Provisional application No. 62/457,557, filed on Feb. 10, 2017, the disclosure of which is incorporated herein by reference.

### BACKGROUND OF THE INVENTION

Long-term (chronic) oxygen deprivation (hypoxia) is a characteristic of several medical conditions, often involving heart and lung. For example, in chronic obstructive pulmonary diseases (COPD), including emphysema and chronic bronchitis which affect more than 5% of US population, lung's ability to extract oxygen from air is severely impaired due to structural and functional damage. This results in chronically low blood oxygen levels in COPD, contributing to premature death and diminished quality of life and mood. Chronic hypoxia is also seen in other medical conditions including chronic mountain sickness, cyanotic heart diseases, cystic fibrosis and obesity. Secondary erythrocytosis (increased red cell mass) usually emerges as a response to blood hypoxia but sustained erythrocytosis is detrimental to health by increasing blood viscosity and risk of thrombosis (coagulation).

Both chronic mountain sickness and COPD patients can benefit from supplemental oxygen. Breathing supplemental oxygen is a life-extending treatment in advanced COPD cases. However, no approaches are currently available that directly target the systemic hypoxia that accompanies COPD or chronic mountain sickness.

### SUMMARY OF THE DISCLOSURE

The present disclosure provides methods and compositions for reducing the systemic effects of chronic hypoxia. For example, a method is provided to reduce the effects of systemic low oxygen conditions. The disclosure is based, at least in part, on the unexpected observation that inhibition of mitochondrial II complex results in reducing the systemic effects of chronic hypoxia.

In one aspect, the method comprises administering to an individual in need of treatment a therapeutically effective amount of a composition comprising one or more of mitochondrial complex II (MTCII) inhibitors. An example of a suitable MTCII inhibitor is atpenin A5. The composition may contain the MTCII inhibitor(s) as the only active agent(s) or may contain other therapeutic agents as well. The administration may be carried out by itself or in conjunction with other therapeutic approaches, such as administration of oxygen or oxygen rich air to the individual.

### BRIEF DESCRIPTION OF THE FIGURES

FIG. 1. Normoxic inhibition of complex II triggers induction of A3A-mediated RNA editing observed in hypoxia. (A) Bar graph depicts percentage SDHB c.136 C>U RNA editing in monocyte-enriched PBMCs (MEPs), approximately 30 million/ml, when treated with Atpenin A5 (AtA5, 1 μM-2 μM) under normoxic (N) or hypoxic (H; 1% O₂) conditions for 1 or 2 days (e.g. H2=day 2 in hypoxia, minimum (n)=4 and maximum (n)=29 donors). (B) Bar graph depicts percentage SDHB c.136 C>U RNA editing upon treatment with TTFA in normoxia for 2 or 3 days. (C) Bar graph depicts percentage SDHB c.136 C>U RNA editing upon treatment with AtA5 and/or IFN1 when subjected to normoxia or hypoxia for 1 or 2 days. Mean and SEM are shown in scatter bar plot. NS: not significant

FIG. 2. Atpenin A5 (AtA5) in normoxia induces transcriptome-scale gene expression responses similar to hypoxia in monocytes (A) Unsupervised heat map shows clustering of hypoxic (day 1) and AtA5/normoxia (N-A) (day 2) samples. Samples 3, 4 and 5 represent CD14 positive monocytes from 3 donors, isolated after culture of MEPs. (B) Scatter plot shows a strong positive correlation between gene expression changes in CD14+ cells upon exposure to hypoxia (day 1) and AtA5/normoxia (day 2) (n=2,131 genes, Pearson r=0.8819, P<0.0001). (C) Bar graph depicts validation of induced expression of selected genes from RNA seq analysis in CD14+ cells under AtA5/normoxia and hypoxic conditions, as determined by RT-qPCR. Inductions by AtA5/normoxia or hypoxia are statistically significant for each gene (p<0.05, Dunnett's multiple comparisons test). (D) Representative immunoblot shows the expression of HIF-1α in lysates (40 μl) of CD14+ and CD14− cells. The cells were isolated after culture for 1 or 2 days in normoxia or hypoxia upon treatment with AtA5 or DMOG. Hypoxia-exposed MEP cells were used to isolate CD14+ and CD14− populations in hypoxia chamber. Actin was used as a loading control (n=3). The immunoblot, performed on the same day, was cropped and merged as depicted by the dotted grey line.

FIG. 3. AtA5 and myxothiazol (MXT) inhibit oxygen consumption and induce A3A-mediated RNA editing. (A) Graph depicts the relative fluorescence levels (mean and SEM with dashed lines), which reflect the degree of hypoxia, on treatment of monocyte-enriched PBMCs (MEPs) with AtA5 or MXT within approximately 3 hours. Control indicates cells without any inhibitors. (B) Bar graph depicts L-Lactate levels in extra-cellular media from the samples analyzed in (A). NS: no significant (C) Bar graph depicts the percentage SDHB c.136 C>U RNA editing upon treatment of MEPs with MXT in normoxia or hypoxia (1% or 6% O₂) for 1 or 2 days. All panels show mean and SEM in MEPs from n=3 donors.

FIG. 4. AtA5 in normoxia induces hypoxic gene expression in monocytes without robust stabilization of HIF-1α. (A) Bar graph depicts fold changes in VEGF and HILPDA gene expression in normoxic and hypoxic CD14+ and CD14− cells upon treatment with AtA5 or MXT for 24 hours (1 day) (n=3 donors). (B) Immunoblot shows the expression of HIF-1α in lysates (40 μl) of CD14+ and CD14− cells examined in (A). The cells were isolated from PBMCs at room conditions followed by culture (5-7 million/ml) for 24 hours in normoxia or hypoxia (1%) upon treatment with AtA5 or MXT. Actin was used as a loading control.

FIG. 5. AtA5 or MXT antagonizes HIF-1α and reduces hypoxic gene expression in transformed cell lines. (A) Immunoblot shows the expression of HIF-1α in lysates (40 μl) of 293T cells upon treatment with DMOG (1 mM), DFO (0.5 mM) and AtA5 (1 μM) when subjected to normoxia or hypoxia (1%) for 24 hours. (B) Immunoblot shows the expression of HIF-1α in lysates (40 μl) of THP-1 cells upon treatment with AtA5 or MXT when subjected to normoxia or hypoxia (1%) for 24 hours (upper panel). Bar graph depicts the fold change in gene expression of HILPDA and VEGFA under the same treatment conditions (n=3 replicates) (lower panel) (C) Immunoblot shows the expression of HIF-1α in lysates (40 μl) of 293T cells upon treatment with AtA5 or MXT when subjected to normoxia or hypoxia (1% or 6%) for 24 hours (upper panel). Bar graph depicts the fold change in gene expression of ELL2 and VEGFA in normoxia and hypoxia (1% O₂) (n=3 replicates) (lower panel) (D) Bar graph depicts the expression of the plasmid expressing luciferase under the control of hypoxia response element (BRE) when 293T cells are treated with AtA5 or MXT in normoxia or hypoxia (1% or 6% O₂) for 24 hours. (n=3 replicates, mean and SEM shown).

FIG. 6. Compound heterozygosity for Sdh subunit null alleles in mice blunts hypoxia-induced increases in hemoglobin levels. (A) Diagram showing Sdhb and Sdhc upstream exons (arrows) splicing into the intronic gene traps (circled). (B) Transgene screening by genomic PCR detects wild-type (WT) homozygous or heterozygous alleles (circles). Arrow shows 600 bp band in 100 bp marker lane. (C) Hemoglobin levels in hypoxia in double (Sdhb/Sdhc) or triple (Sdhb/Sdhc/Sdhd) heterozygous mice are consistently lower than in wild-type controls (p<0.05, Fisher's combined probability test (68), n=4 or 5 male with similar age in each cohort, Mean and SEM).

FIG. 7. Mice with partial Sdh defects live longer under chronic lifelong hypoxia compared to wild type mice. Three independent cohorts of mice (n=4 or 5 in each cohort) show consistently higher survival in Sdh transgenic mice relative to wild type mice. Combined analysis shows a statistically significant increase (about 12% increase relative to wild type mice) in survival of Sdh mice under chronic hypoxia. Mice are placed in hypoxia chamber from weaning until they spontaneously die or develop morbidities that justify euthanasia according to rules set forth by Roswell Park Cancer Institute transgenic facility under an approved IACUC protocol. Also see methods in accompanying paper in press.

FIG. 8. Survival curves for Sdh defect mice from FIG. 7 are significantly different from those of wild type mice. Table showing a comparison of survival between WT and Sdh mice (all three cohorts combined).

FIG. 9. Top gene clustering shows similar gene expression changes in hypoxic versus Atpenin A5 (AtA5)/normoxia (N-A) CD14+ cells. (n=3 donors)

FIG. 10. Atpenin A5 (AtA5) in normoxia induces hypoxia-related SDHB c.136C>U RNA editing and gene expression in CD14+ monocytes in three additional donors. (A) Bar graph depicting SDHB c.136 C>U RNA editing in CD14+ and CD14− cells upon AtA5 treatment when subjected to normoxia or hypoxia (1%) for 1 or 2 days. (B) Bar graph depicting fold change in ELL2, HILPDA and VEGFA under similar conditions as (A) in CD14+ cells. (C) Bar graph depicting the fold change in gene expression of ELL2, HILPDA and VEGFA upon treatment with AtA5 under normoxia or hypoxia in MEPs for 24 hours (n=3, mean and SEM are shown).

FIG. 11. Bar graph depicting SDHB c.136 C>U RNA editing in MEPs upon myxothiazol (MXT) treatment when subjected to normoxia or hypoxia (1%) for 24 hours. (n=3, mean and SEM are shown).

FIG. 12. Bigger images of western blots in FIG. 2D and FIG. 4B are shown. Note the accessory bands detected by actin antibody in some samples.

FIG. 13. Listing of PCR oligonucleotide primers (5′ to 3′).

FIG. 14. Red blood cell indices derived from complete blood counts, including red blood cell distribution width (RDW-SD) (b), reticulocyte fraction (c) and immature reticulocyte fraction (IRF) (d) measured by Procyte Dx, are shown for Sdhb/c/d triple het versus wild type littermate controls (n=5 each group, mean and SEM are shown). Also shown are consistent but statistically insignificant reduction in white cell counts (a) in Sdh mice relative to control. Summary P values, which combine individual P values for each time point by Fisher's Chi square method, are shown. P values for individual time points where SEM bars do not overlap are also statistically significant.

FIG. 15. Total body weight at the time of death is statistically significantly higher in Sdh mice than in wt controls (p=0.0159, Mann-Whitney test, 2-sided). No statistically significant difference is seen between Sdh and wt mice in normoxia.

### DETAILED DESCRIPTION OF THE INVENTION

Abbreviations

A3: APOBEC3

AtA5: atpenin A5

HIF: hypoxia-inducible factor

IFN1: interferon type 1

MEPs monocyte-enriched PBMCs

MXT: myxothiazol

MTCII: mitochondrial complex II

PBMC: peripheral blood mononuclear cells

PGL: paraganglioma tumor

RPKM: reads per kilobase of transcript per million mapped reads

SDH: succinate dehydrogenase

SEM: standard error of mean

Wt or wt: wild type

The terms “systemic hypoxia” or “systemic low oxygen condition” are used interchangeably and mean hypoxic conditions affecting essentially the entire body. Hypoxia can be measured clinically. For example, arterial oxygen tension is one way to measure hypoxia. Arterial blood oxygen is usually measured by blood-gas analyzers in laboratory or at point of care. An arterial oxygen tension of 80-100 mm Hg is considered normal. An arterial oxygen tension of 60-79 mm Hg is considered mild hypoxia, 40-60 mm Hg is considered medium hypoxia and less than 40 mm Hg is considered to be severe hypoxia. The systemic hypoxia condition may be acute (generally lasting a few seconds or hours) and subacute (generally lasting days to weeks) or chronic (generally over a period of a month or longer).

The present disclosure provides a method to alleviate the effects of chronic hypoxia by inhibition of MTCII complex. The present method can be used for mild, medium or severe hypoxia. The inhibition of MTCII may be complete or partial. When partial, the inhibition may be from 1% to 99% and all percentages and ranges therebetween. For example, the inhibition may be from 5% to 95% and all percentages and ranges therebetween, including. The inhibition may be 10%, 20%, 30%, 40%, 50%, 60%, 70%, 80%, or 90%. The chronic hypoxia may manifest as systemic hypoxia, which can be persistent or episodic. The present method can be used for treating any medical condition which is accompanied by systemic hypoxia (persistent or episodic), including, but not limited to, COPD, cyanotic heart diseases, cystic fibrosis, congestive heart failure, pulmonary embolism, asthma, idiopathic pulmonary fibrosis, acute respiratory distress syndrome and the like. The present method can be used for one or more of the following: to blunt levels of secondary erythrocytosis, to prolong survival in chronic hypoxia, suppress secondary polycythemia, suppress hemoglobin levels, and/or suppress any other symptom or condition associated with chronic hypoxia.

An example of an MTCII inhibitor is atpenin A5 (3-((2S,4S,5R)-5,6-dichloro-2,4-dimethylhexanoyl)-2-hydroxy-5,6-dimethoxypyridin-4(1H)-one). Other examples include malonate, diazoxide (DZX), malate and oxaloacetate, 3-nitropropionic acid, nitroxyl, carboxin, TTFA (thenoyltrifluoroacetone) and lonidamine.

In one aspect, the present disclosure provides a composition for use in the treatment of chronic systemic hypoxia. The composition comprises a MTCII inhibitor and a pharmaceutical carrier. For example, the composition can comprise Atpenin A5. The MTCII inhibitor (such as atpenin A5) may be the only active agent in the composition or there may be other active agents. For example, atpenin A5 may be the only agent in the composition that has any effect on the mitochondrial complex II.

The present disclosure is based on the unexpected observation that inhibition of mitochondrial complex II resulted in reducing the effects of chronic systemic hypoxia. While not intending to be bound by any particular theory, it is considered that the present method of inhibition of MTCII complex for a condition associated with systemic hypoxia, may reduce the systemic need for oxygen or reduce the amount of oxygen required by an individual afflicted with a systemic low oxygen condition.

The present method comprises administering to the individual in need of treatment a composition comprising or consisting essentially of a therapeutically effective amount of one or more MTCII inhibitors. Administration of the inhibitor may result in suppressing hemoglobin levels, reducing red cell distribution width (RDW) and/or prolong survival and life expectancy.

The composition comprising the MTCII inhibitor may contain other active agents, or the MTCII inhibitor may be the only active agent in the composition. The compositions will generally contain pharmaceutical carriers. Examples include, but are not limited to, saline, buffered saline, dextrose, water, glycerol, ethanol etc.

In one embodiment, the compositions do not contain mitochondrial complex III (MTCIII) complex inhibitors.

The individual in need of treatment can be a mammal, including humans and non-human mammals. Non-human mammals treated using the present methods include domesticated animals (e.g., canine, feline, murine, rodentia, and lagomorpha) and agricultural animals (e.g., bovine, equine, ovine, porcine).

The phrase “treating” or “treatment” as used herein means reducing the severity of one or more of the symptoms associated with the indication that the treatment is being used for. Thus treatment includes ameliorating one or more symptoms associated with an indication.

The term “therapeutically effective amount” of a compound (e.g., MTCII inhibitor) refers to an amount which is effective, upon single or multiple dose administration to an individual, for alleviating the symptoms of, or treating the particular indication. The exact amount desired or required will vary depending on the particular compound or composition used, its mode of administration, patient specifics, and the like. Appropriate effective amount can be determined by one of ordinary skill in the art informed by the instant disclosure using only routine experimentation. As an example, the dosage of MTCII inhibitor, such as atpenin A5, can be such that the systemic exposure of cells is to a concentration of about 0.05 μM to 500 μM or about 0.1 μM to 500 μM and all values therebetween to the tenth decimal place, including and from 0.05 μM to 500 μM or 0.1 μM to 500 μM. For example, the cells may be exposed to about 0.05 μM to 50 μM, or 0.1 μM to 50 μM, or 1 μM to 50 μM atpenin A5 and all values therebetween. In embodiments, the cells may be exposed to 1, 5, 10, 50, 100, 250, 400, or 500 μM atpenin A5. It will be appreciated that the concentration that the cells are exposed to may not be constant and may fluctuate. In one embodiment, the concentration of Atpenin A5 that the cells are exposed to is kept within a range of 0.05 μM to 500 μM over a desired period of time. The MTCII inhibitor may be administered as pharmaceutically acceptable salt and may be delivered in pharmaceutically acceptable carriers including liquid or solid filler, diluent, excipient, solvent or encapsulating material, involved in carrying or transporting the subject chemical from one organ, or portion of the body, to another organ, or portion of the body. For example, compositions comprising MTCII inhibitor can be provided in liquids, caplets, capsules, tablets, inhalants or aerosol, etc. Delivery devices may comprise components that facilitate release over certain time periods and/or intervals, and can include compositions that enhance delivery of the pharmaceuticals. For example, nanoparticle, microsphere or liposome formulations can be used. The compositions described can include one or more standard pharmaceutically acceptable carriers. Examples of pharmaceutically acceptable carriers can be found in: Remington: The Science and Practice of Pharmacy (2005) 21st Edition, Philadelphia, Pa. Lippincott Williams & Wilkins. In one embodiment, the amount of MTCII inhibitor per administered dose can be from 0.05 mg/kg to 5.0 mg/kg body weight (and all values therebetween to the tenth decimal point). For example, the amount of MTCII inhibitor per administered dose can be 0.05, 0.1, 0.25, 0.5, 1.0, 2.5, 3.5, 4.5 or 5 mg.kg body weight. For example, the amount can be 1.0 mg/kg, which may be given orally or parenterally.

Treatment with a MTCII Inhibitor can be continued as long as the individual is experiencing hypoxia. In conditions such as COPD, the treatment can be life-long treatment. The treatment can be continuous or intermittent. Treatment effectiveness can be monitored by measuring hemoglobin levels, RDW or other symptoms associated with chronic systemic hypoxia. In one embodiment, a continued reduction in one of more symptoms is indicative of the effectiveness of the treatment. Monitoring of various parameters related to chronic systemic hypoxia or the effects of MTCII treatment (including arterial blood oxygen, hemoglobin levels, RDW) can be measured prior to initiation of the treatment, during the treatment regimen, and/or after termination of the treatment.

The present compositions can be administered via any of the known methods in the art. For example, the compositions can be administered orally, parenterally, sublingually, transdermally, rectally, transmucosally, topically, via inhalation, via buccal administration, or combinations thereof. Parenteral administration includes, but is not limited to, intravenous, intraarterial, intracranial, intradermal, subcutaneous, intraperitoneal, subcutaneous, intramuscular, intrathecal, and intraarticular. The MTCII inhibitors can also be administered in the form of an implant, which allows a slow release of the inhibitors, as well as by slow controlled i.v. infusion.

The basis for the present disclosure is at least in part the following observations. We used a hypobaric hypoxia chamber to study the effects of long term hypoxia in mice. The oxygen concentration in the chamber was about 10%, roughly corresponding to 6,000 altitude-meters. We found that mice with a specific genetic defect in mitochondria live longer under chronic life-long hypoxia compared to wild-type control mice. Mitochondria are intracellular organelles that consume oxygen to produce energy. Complete inhibition of oxygen consumption by mitochondria is lethal. However, we found that partial inhibition of MCTII (also known as succinate dehydrogenase; Sdh) by compound heterozygous mutations in two (Sdhb/Sdhc) or three (Sdhb/Sdhc/Sdhd) Sdh subunit genes is compatible with survival under normal oxygen levels (21%) but blunts secondary erythrocytosis and prolongs survival by 10%-15%.

We identified two molecular consequences of inhibition of complex II: (1) induction of gene expression changes for hypoxia adaptation in certain cell types such as peripheral blood monocytes; (2) reduction of oxygen consumption and suppression of activities of hypoxia induced transcription factors (called HIF1 and HIF2), as demonstrated by reduced hemoglobin levels in the complex II transgenic mice in chronic hypoxia. It is known that persistent activation of HIFs can be detrimental to life. Thus, in the present disclosure, it is considered that these two factors (increased adaptation of certain cell types to hypoxia and suppression of HIFs) may independently or in combination help prolong survival in hypoxia upon partial inactivation of mitochondrial complex II. Reduced levels of secondary erythrocytosis, which is mainly regulated by HIF2, may be involved in prolonged survival. The effect of mitochondrial complex II inhibition on survival in hypoxia is surprising and unexpected. Our observations indicate that patients with chronic hypoxia can benefit from pharmacologic inhibition of mitochondrial complex II for increased longevity. Inhibition of complex II may also be used to reduce red blood cell mass in secondary erythrocytosis.

It is considered the present method involves improving the altered oxygen supply/demand relationship in conditions of chronic hypoxia and is based on reducing organismal oxygen demand. While oxygen supplementation is the traditional method to improve systemic oxygenation, it is considered the present method may suppress systemic oxygen consumption by partially inhibiting mitochondria. Complete blockage of respiration (as seen with cyanide) is lethal due to halting of oxygen consumption. In contrast, in the present disclosure inhibition of mitochondrial II complex partially reduces mitochondrial oxygen consumption, which may reduce HIF activity in hypoxia. It is considered that this also stimulates hypoxia adaptation pathways in certain cells such as blood monocytes. These pathways, triggered by inhibition of mitochondrial complex II may combine to prolong survival under systemic hypoxia conditions.

The present method may be used as a complementary approach to supplemental oxygen administration in conditions of chronic systemic hypoxia, including COPD or chronic mountain sickness.

The following examples are provided as illustrative of the present methods. These examples are not intended to be restrictive in any way.

### EXAMPLE 1

In this example we demonstrate that inhibition of MTCII mimics the effects of hypoxia. We observed that inhibition of MTCII mimicked transcriptional effects of hypoxia in normoxic monocytes without robust stabilization of HIF-1α, but antagonizes (a) hypoxic stabilization of HIF-1α in transformed cell lines and (b) hypoxia-induced increases in hemoglobin levels in a heterozygous Sdh mouse model. Several earlier studies in transformed cell lines suggested that normoxic stabilization of HIF-1α explains the persistent expression of hypoxic genes upon complex II inactivation. On the contrary, we find that atpenin A5 antagonizes the stabilization of HIF-1α and reduces hypoxic gene expression in transformed cell lines. Accordingly, compound germline heterozygosity of mouse Sdhb/Sdhc/Sdhd null alleles blunts chronic hypoxia-induced increases in hemoglobin levels, an adaptive response mainly regulated by HIF-2α. In contrast, atpenin A5 or myxothiazol does not reduce hypoxia-induced gene expression or RNA editing in monocytes. These results reveal a novel role for mitochondrial respiratory inhibition in induction of the hypoxic transcriptome in monocytes and indicates that inhibition of complex II activates a distinct hypoxia signaling pathway.

Results

Atpenin A5 (AtA5) in Normoxia Induces Hypoxia-Related RNA Editing by A3A in Monocytes

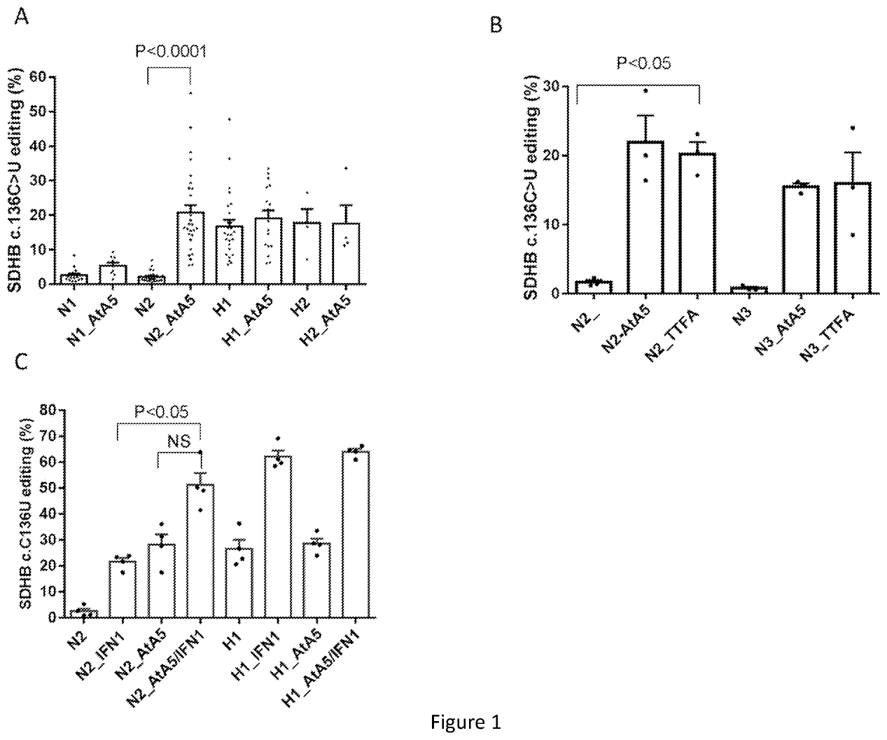

To test whether inactivation of MTCII triggers hypoxia responses in monocytes, we used AtA5, a ubiquinone homolog and a highly specific and potent inhibitor. AtA5 in normoxia (AtA5/normoxia) induced SDHB c.C136U RNA editing, especially on day 2 in cultures of monocyte-enriched PBMCs (MEPs) (FIG. 1A). RNA editing levels induced by hypoxia (day 1) versus AtA5/normoxia (day 2) were similar. Joint treatment by AtA5 and hypoxia did not further increase RNA editing levels. TTFA, another ubiquinone analog but a less potent inhibitor of MTCII, also induced RNA editing in normoxia (FIG. 1B). A3A-mediated RNA editing by hypoxia and IFN1 is additive (30). We find that RNA editing by AtA5 and IFN1 in normoxia is also additive (FIG. 1C), whereas no additional effect of AtA5 is seen in hypoxia with IFN1. These results demonstrate that normoxic inhibition of MTCII induces A3A-mediated RNA editing in monocytes in a manner similar to hypoxia.

AtA5 in Normoxia Induces Hypoxia-Related Gene Expression in Monocytes

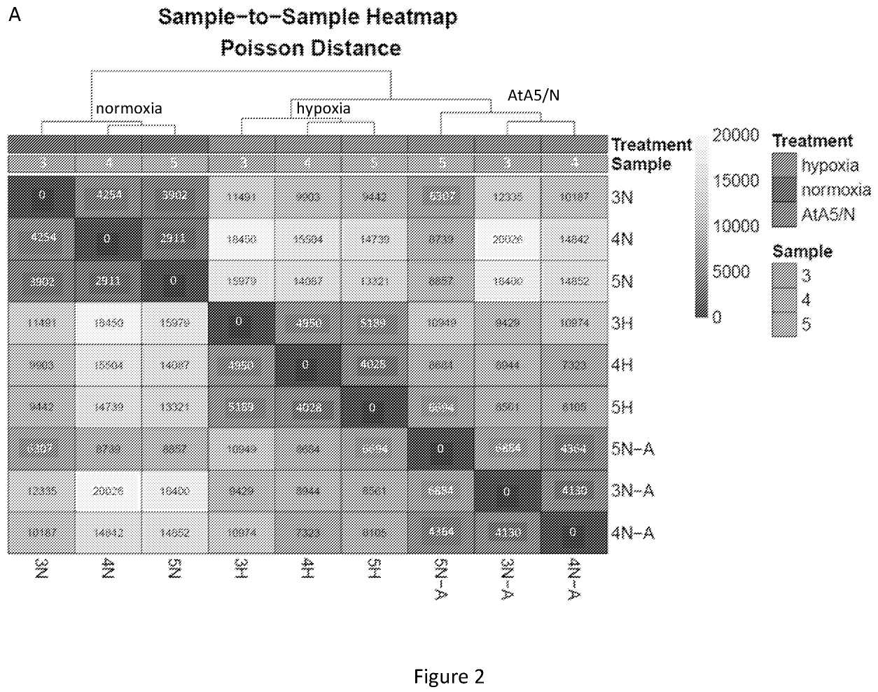

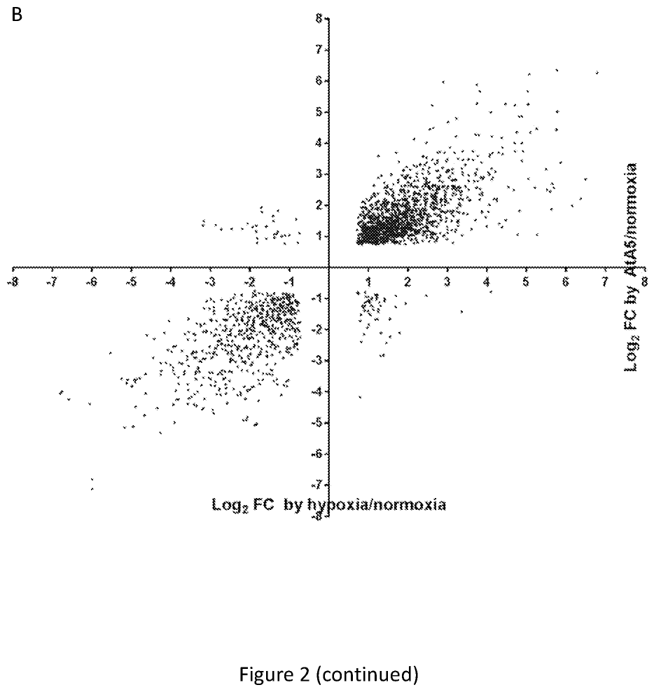

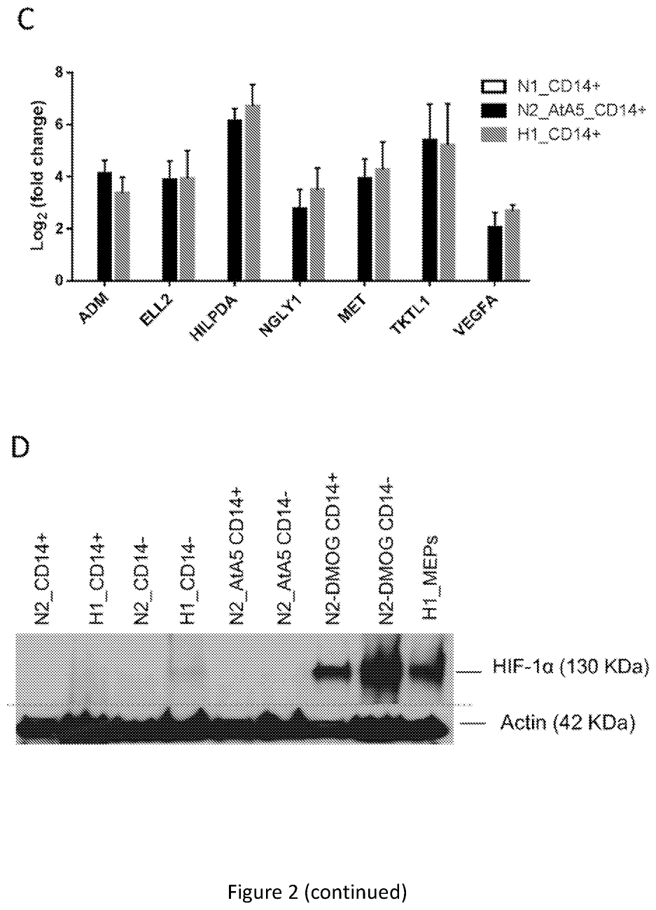

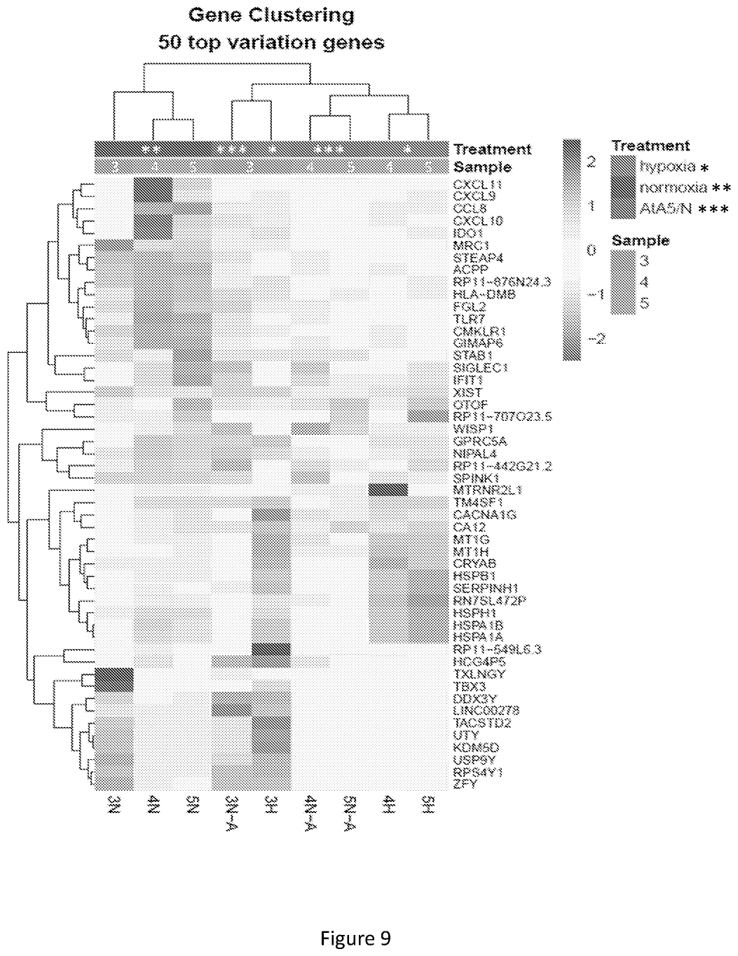

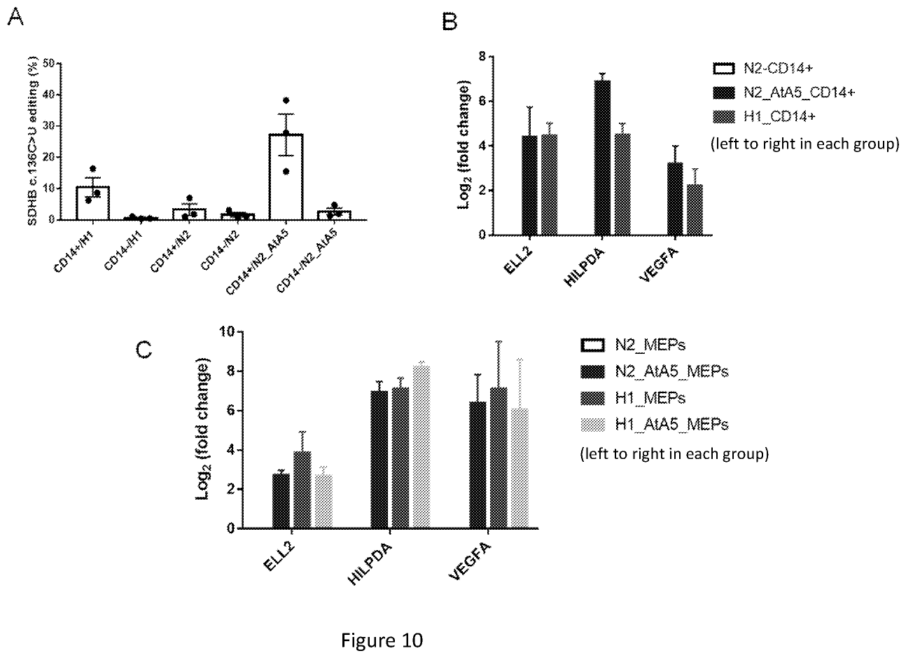

We examined whether MTCII also regulates induction of gene expression in primary monocytes under hypoxia or when inactivated, as observed in SDH-mutated paragangliomas. To test the impact of MTCII inhibition on monocyte gene expression, we cultured MEPs (n=3 donors) in normoxia (1 day), hypoxia (1 day) and AtA5/normoxia (2 days), isolated CD14+ monocytes and performed RNA seq analysis. SDHB c.136C>U RNA editing increased in CD14+ cells in hypoxia (mean±SEM=29.9%±9.9%) and AtA5/normoxia (mean±SEM=32.1%±14.9%) relative to normoxic controls (mean±SEM=5.4%±1.7%). Gene expression data without making any assumptions on the experimental design revealed evidence of similar expression patterns with hypoxia and AtA5/normoxia treatment (FIG. 2A and FIG. 9). Genes that are expressed at high levels (RPKM>0.5, n=9,389) and statistically significantly affected by hypoxia or AtA5/normoxia (p-adjusted <0.05) showed a very strong positive correlation in fold changes (FIG. 2B). HIF1A but not EPAS1 (HIF2A) or HIF3A is expressed robustly (RPKM averages 21.5, 0.17 and 0.01, respectively) in monocytes. Hypoxia or AtA5/normoxia induced RNA editing without transcriptional upregulation of A3A RT-qPCR analyses confirmed the induction of selected highly-expressed genes (ADM, ELL2, HILPDA, NGLY1, MET, TKTL1 and VEGFA) both by hypoxia and AtA5/normoxia (FIG. 2C). RNA seq analysis showed induction of RNA editing in genes other than SDHB by both hypoxia and AtA5. The induction of SDHB mRNA editing and hypoxic gene expression are confirmed in CD14+ monocytes obtained from 3 additional donors as well as in MEPs by hypoxia at day 1 and by AtA5/normoxia at day 2 but not in untreated normoxic controls at day 2 (FIG. 10). Western blot showed HIF-1α stabilization in hypoxic (1% O₂) CD14− cells but not in hypoxic CD14+ monocytes nor in normoxic CD14+ or CD14− cells treated with AtA5 (FIG. 2D). DMOG, an inhibitor of PHD enzymes, caused robust normoxic stabilization of HIF-1α both in CD14+ and CD14− cells (day 2). These results suggest that hypoxia and AtA5/normoxia have similar signaling pathways to induce the gene expression changes in CD14+ monocytes independently of HIF-1α.

AtA5 and Myxothiazol Inhibit Oxygen Consumption and Induce Hypoxia Responses in Monocytes without Robust Stabilization of HIF-1α

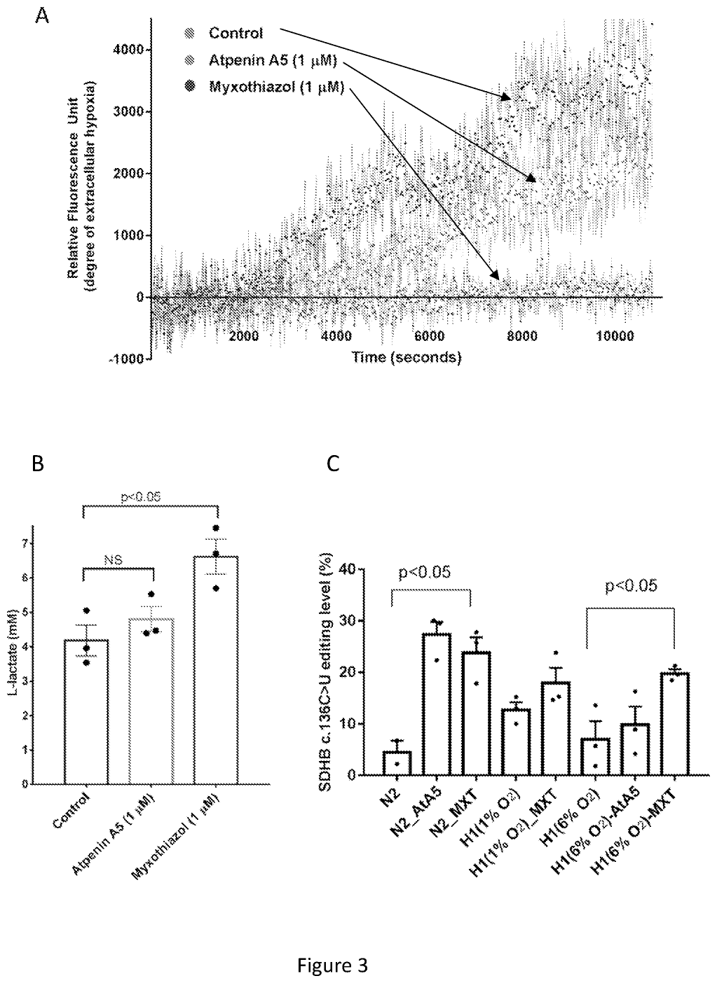

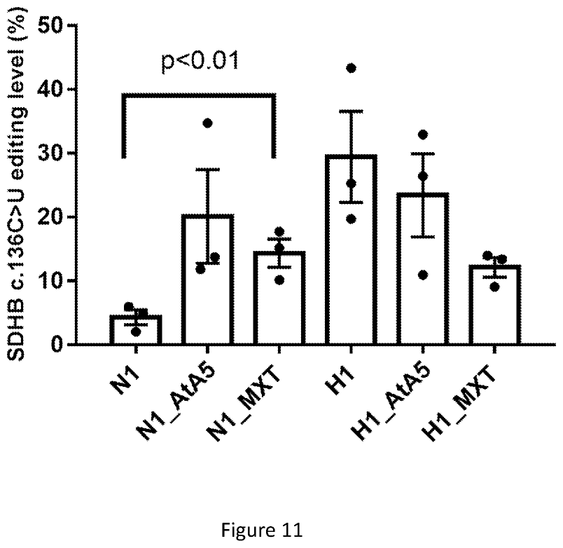

To examine whether hypoxia responses in monocytes are also induced by the inhibition of another mitochondrial complex, we used myxothiazol (MXT), a ubiquinole analog which inhibits complex III. We first measured oxygen consumption and L-lactate levels to confirm the effect of AtA5 and MXT on mitochondrial respiration in MEPs. We used a phosphorescent oxygen probe (MitoXpress-Xtra) which is quenched by oxygen. Cellular respiration in a closed system depletes oxygen and increases the fluorescence. We find that complex III inhibitor MXT completely suppressed oxygen consumption (FIG. 3A), whereas AtA5 reduced but did not abolish it in primary cells (MEPs). L-Lactate, the end product of glycolysis, increased relative to the degree of respiratory inhibition (FIG. 3B). MXT induced SDHB mRNA editing in normoxia and increased the editing level in mild hypoxia (6% O₂), but had no statistically significant effect on it at 1% O₂ when compared to hypoxia (1% O₂) alone (FIG. 3C). Normoxic induction of RNA editing by MXT is also confirmed in a separate experiment from three additional donors (FIG. 11).

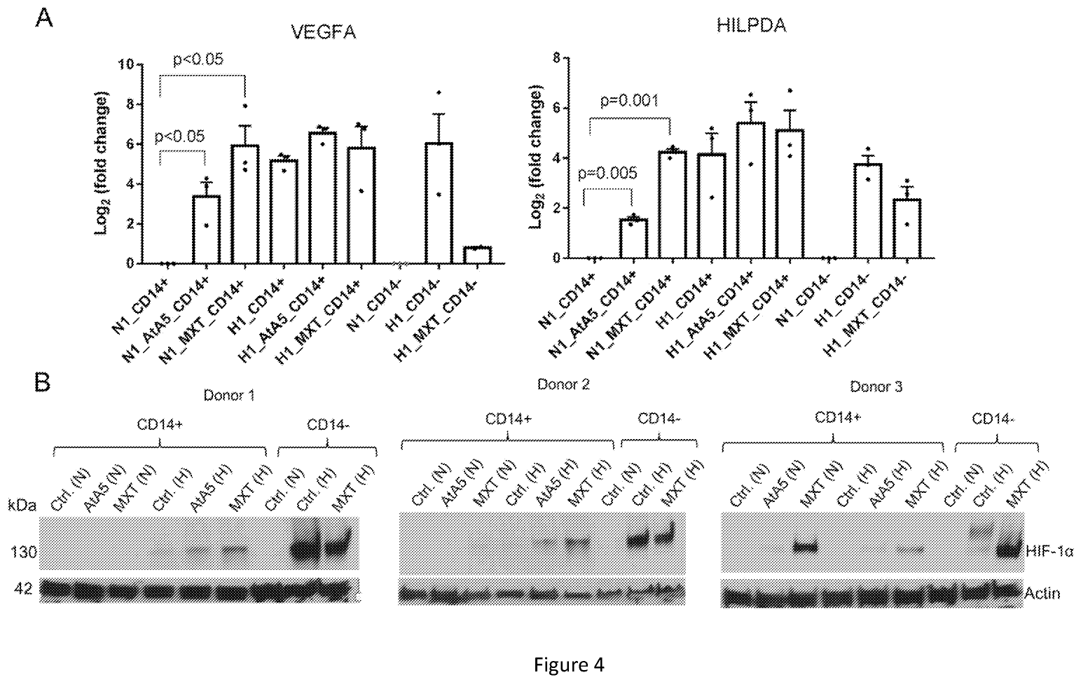

To further examine the effect of MXT and AtA5 on the expression of hypoxia regulated genes and HIF-1α protein expression, we first isolated CD14+ and CD14− cells from PBMCs of three additional donors and then exposed them for 1 day to normoxia or hypoxia (1% O₂) with or without the inhibitors. MXT and AtA5 statistically significantly increased the mRNA expression of VEGFA and HILPDA in CD14+ monocytes in normoxia but not in hypoxia (FIG. 4A). Hypoxia showed robust stabilization of HIF-1α in CD14− but not in CD14+ cells (FIG. 4B). Similarly, AtA5 and MXT did not robustly stabilize HIF-1α in normoxia in monocytes. AtA5 and MXT appeared to enhance the stabilization of HIF-1α in hypoxic monocytes in two of three donors (FIG. 4B). Thus, hypoxia or mitochondrial inhibitors in normoxia induce hypoxic gene expression in monocytes without consistent stabilization of HIF-1α when compared to its robust stabilization in hypoxic lymphocytes.

AtA5 and MXT Suppress HIF-1α and Hypoxia-Induced Gene Expression in Cell Line

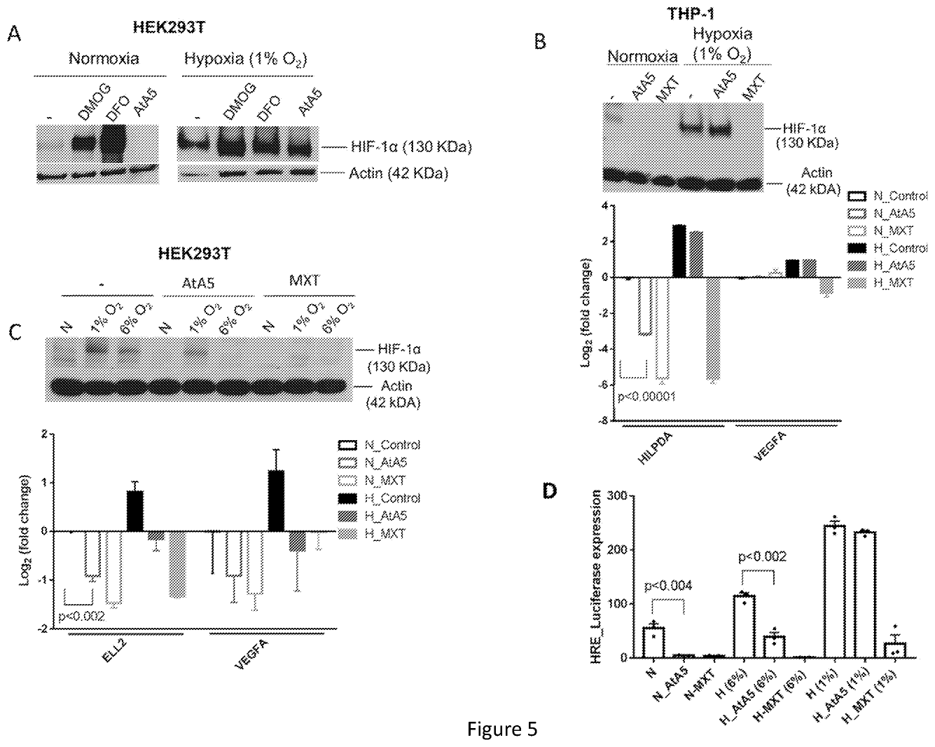

Our results so far suggest that normoxic inhibition of complex II or complex III in monocytes induces hypoxia responses, both RNA editing and gene expression, without consistent stabilization of HIF-1α. It is possible that HIF-1α may have degraded depending on cell type, time of analysis (24 h) or another factor. Therefore, we further examined the effect of AtA5 in HEK293T embryonic kidney cell line and THP-1 monocytic leukemia cell line over a 24 hour period. Several studies in cell lines have reported normoxic stabilization of HIF-1α upon knocking down MTCII (Selak et al., (2005), Cancer. Cell, 7, 77-85, Guzy et al., (2008), Mol. Cell. Biol., 28, 718-731, Cervera et al., (2008), Cancer Res., 68, 4058-4067). We found that DMOG and DFO, but not AtA5 stabilized HIF-1α in normoxia in HEK293T cells. Moreover, AtA5 suppressed the weak HIF-1α expression in normoxia, which was possibly seen due to cellular crowding and peri-cellular hypoxia (FIG. 5A). Similarly, AtA5 antagonized weak normoxic stabilization of HIF-1α in THP-1 cells, whereas MXT inhibited it even at 1% O₂ (FIG. 5B). Since AtA5 appeared to suppress HIF-1α in mild hypoxia, we exposed the cells to both 6% and 1% O₂. Confirmatory experiment in 293T cells showed that AtA5 inhibited stabilization of HIF-1α only at 6% O₂, whereas MXT inhibited it both at 6% O₂ and 1% O₂ (FIG. 5C). RT-qPCR analyses showed that expression of hypoxia-regulated genes is suppressed by AtA5 and MXT in parallel to their ability to suppress HIF-1α (FIGS. 5B and 5C, lower panels). To confirm the suppression of HIF-regulated gene expression, we used a plasmid construct expressing firefly luciferase under the control of three hypoxia-response elements (EIRE) from PGK1, a HIF-1α target gene. AtA5 statistically significantly suppressed HRE-driven expression in 6% O₂ but not in 1% O₂, whereas MXT inhibited HRE-driven expression both in 6% O₂ and 1% O₂ (FIG. 5D). These results demonstrate that inhibition of MTCII by AtA5 in transformed cell lines does not stabilize HIF-1α in normoxia, but rather blocks it in mild hypoxia.

MTCII Mutations Reduce Hemoglobin Levels in Chronically Hypoxic Mice

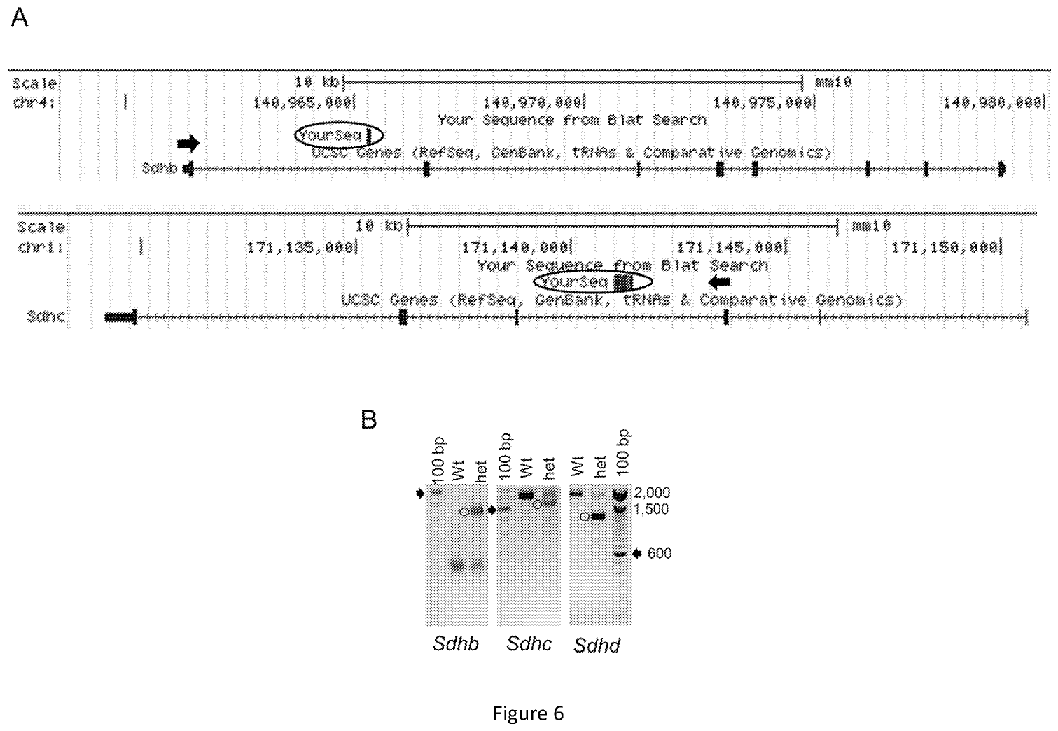

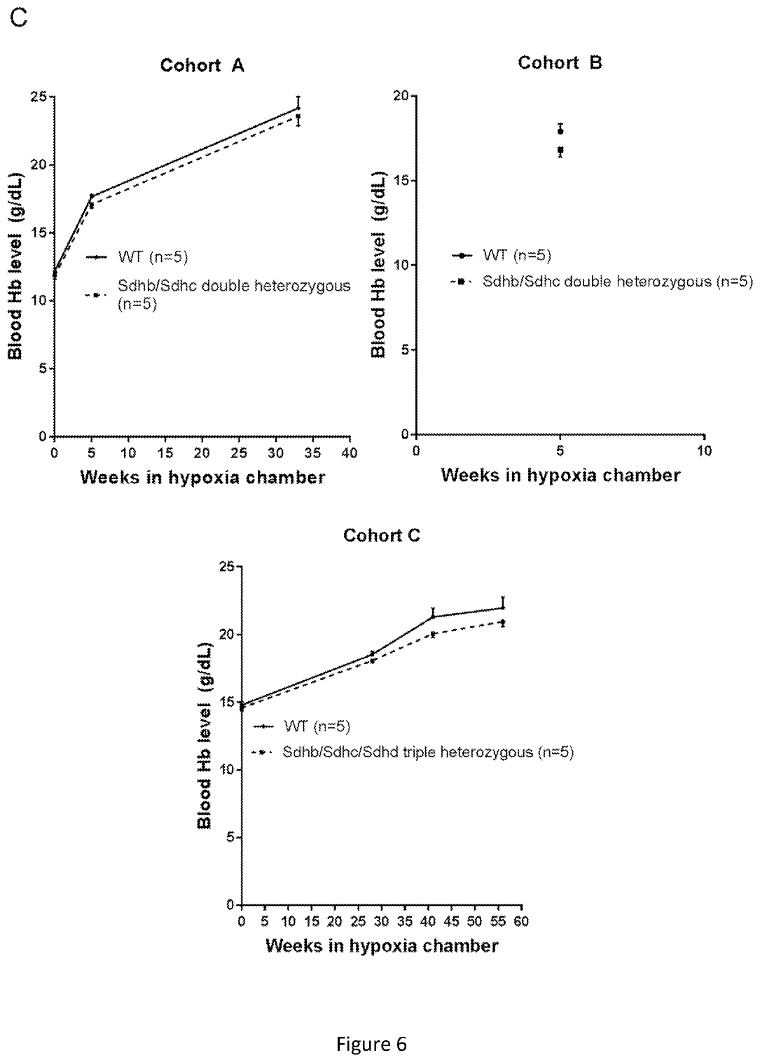

Since AtA5 does not induce HIF-1α in normoxia but appears to antagonize its hypoxic stabilization in 293T and THP-1 cell lines, we further studied the impact of MTCII inhibition on hypoxia response in vivo. Mice with Sdhb, Sdhc and Sdhd heterozygous knockout alleles were cross-bred to obtain Sdhb/Sdhc double heterozygous and Sdhb/Sdhc/Sdhd triple heterozygous mice. Sdhb, Sdhc, and Sdhd are located on mouse chromosomes 4, 1, and 9, respectively. Cross-mating of Sdhb/Sdhc double heterozygous mice did not give any viable progeny homozygous for Sdhb or Sdhc mutations (p<0.0001, Chi-Square test), supporting that Sdhb and Sdhc alleles obtained by gene trapping are null (FIGS. 6A and B). Mating of Sdhb/Sdhc double heterozygous mice with Sdhd heterozygous mice produced all possible genotypes including Sdhb/Shdc/Sdhd triple heterozygosity as expected from independent Mendelian segregation (p=0.5161, Chi-Square test). To examine the role of Sdh on hemoglobin levels, we exposed mice to chronic hypobaric hypoxia (9%-10% O₂). Hypoxia-induced increases in hemoglobin levels were less in Sdh mutant mice compared to the WT control in each six measurement performed in three cohorts (range=2.4% to 6%) (FIG. 6C). Although the effect size was small in each measurement (p>0.05), combined analysis was statistically significant to support the hypothesis that partial inactivation of Sdh antagonizes hypoxia-induced increases in Hb levels (P<0.05, FIG. 6C). Since hypoxia-induced increases in Hb levels are thought to be mediated by HIFs, primarily HIF-2, these in vivo results suggest that inactivation of MTCII antagonizes HIF-mediated responses to hypoxia.

MTCII Mutations Prolong Survival Time Under Chronic Hypoxic Conditions

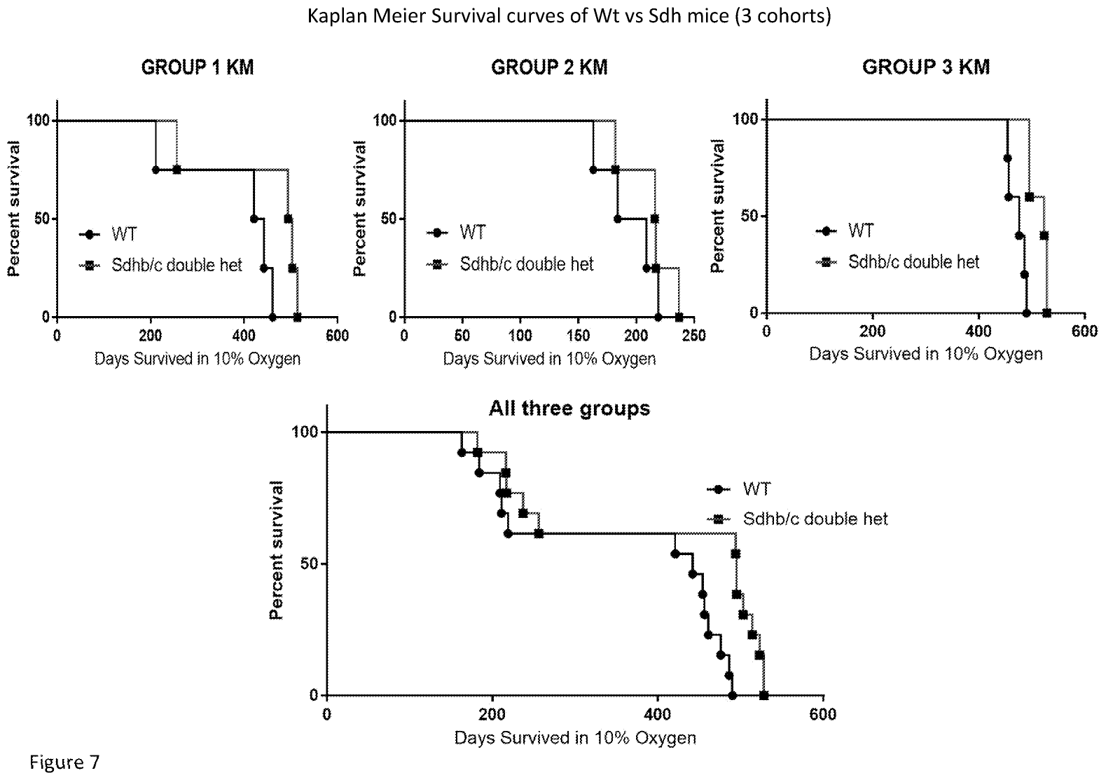

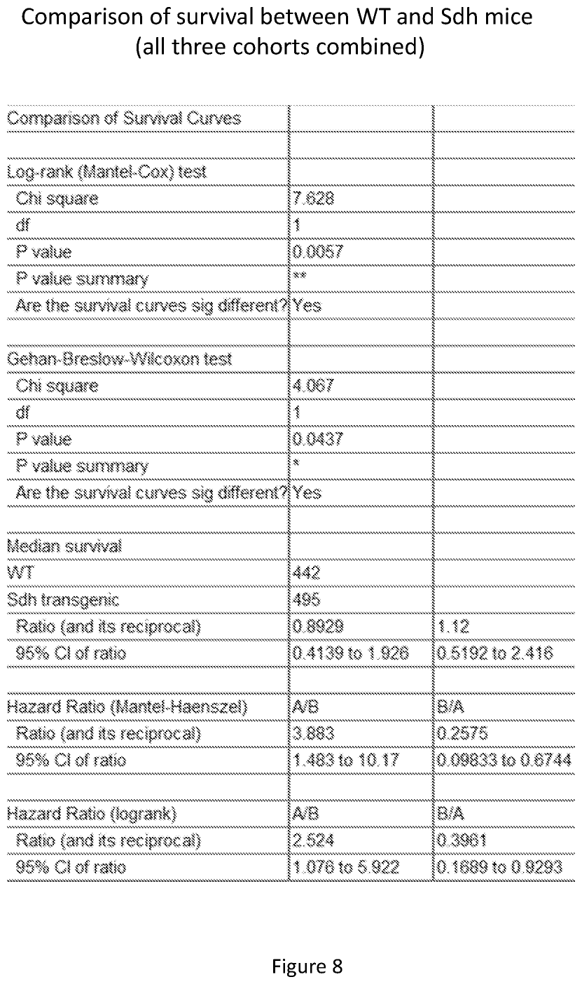

Sdh transgenic mice and wild type mice were exposed to chronic hypoxic conditions. FIGS. 7 and 8 shows that mice with partial Sdh (complex II) defects live longer under chronic lifelong hypoxia compared to wild type mice. The precise mechanisms underlying this extended survival in the transgenic mice is unknown. However, certain characteristics of the transgenic mice including reduced hemoglobin levels, reduced red cell distribution width (RDW) and higher body weight at the time of death may all contribute to prolonged survival under chronic hypoxia. Furthermore, inhibition of MTCII may activate hypoxia adaptation pathways in certain cell types like monocytes.

Sdh Transgenic Mice Show Additional Alterations

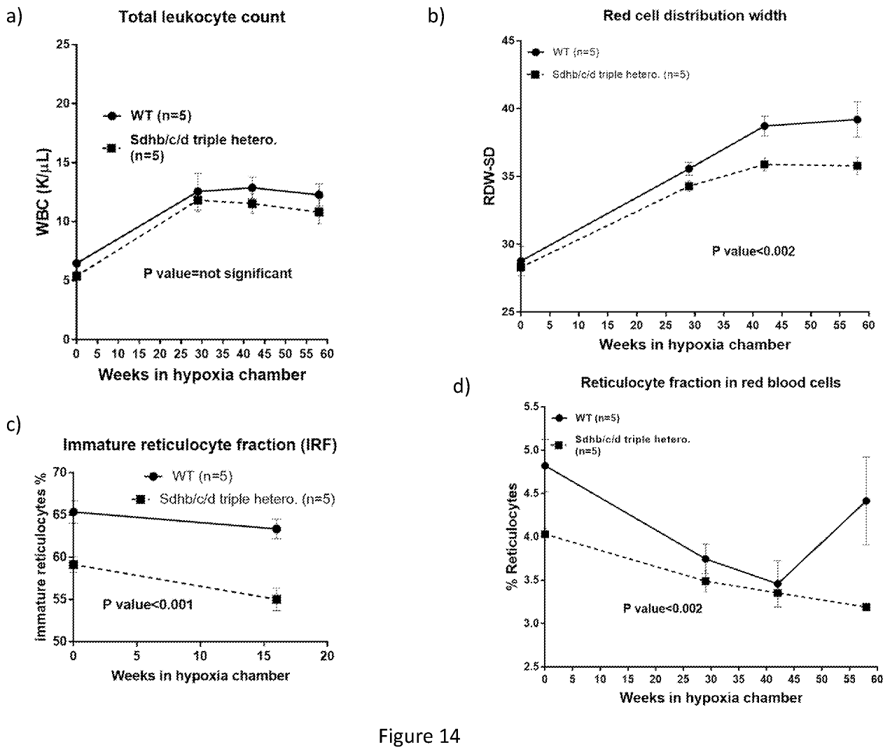

Further studies were carried out to analyze additional blood parameters independent of Hb levels in mice with MTCII mutations and wild type mice. Blood was collected from these mice and total leukocyte count, red cell distribution width (RDW), immature reticulocyte fraction (IRF), and reticulocyte fraction in red blood cells was determined. Results are shown in FIG. 14. Reduction in RDW (red blood cell distribution width, (b)), immature reticulocyte fraction (c), reticulocyte percentage (d), and related red blood cell parameters such as ratio of most immature to most mature reticulocyte fractions were observed. These findings are consistent with reduced red cell production activity in Sdh mice, which may be related to lower erythropoietic stimulation, lower systemic inflammation or other mechanisms relative to wt mice.

High RDW is associated with overall mortality in acute and chronic conditions, cardiovascular disease, venous thromboembolism, cancer, diabetes, community-acquired pneumonia, chronic obstructive pulmonary disease, liver and kidney failure (Lippi et al 2009, Archives of pathology & laboratory medicine. April 133(4):628-32; Salvagno et al 2015, Critical reviews in clinical laboratory sciences, March 4; 52(2):86-105). Our chronic hypoxia mouse model indicates that suppression of mitochondrial complex II reduces RDW which is therapeutically relevant particularly in respiratory and circulatory conditions that are associated with high hypoxic burden.

We also observed consistent reductions in total white blood cell (WBC) count in Sdh mice relative to wt control (a), but the differences are not statistically significant.

Alterations in the above-mentioned CBC indices in Sdh transgenic mice indicates that efficacy of any drug or drug-like compound that inhibits Sdh (mitochondrial complex II) can be monitored by their effect via these blood indices under normoxic or hypoxic conditions.

Sdh Transgenic Mice Have Higher Body Weight Than Wild Type Controls

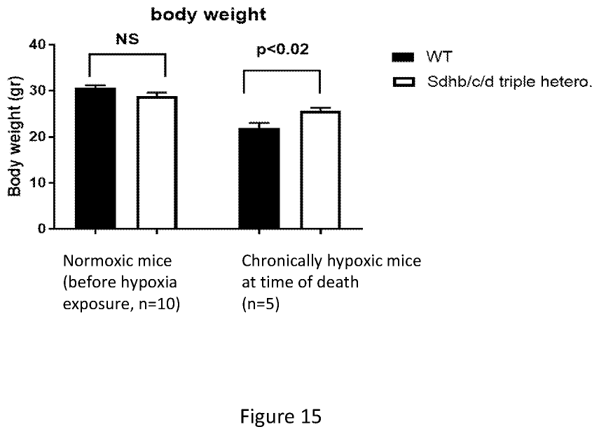

Sdh transgenic mice and wild type mice were subjected to chronic hypoxia or normoxia as described above and total body weight was measured at the time of death. As shown in FIG. 15, total body weight at the time of death is statistically significantly higher in Sdh mice than in wt controls. In contrast, no statistically significant difference is seen between Sdh and wt mice in normoxia. The average weight of normoxic control mice (both wt and Sdh) was higher than the average weight of chronically hypoxic mice at the time of death (29.75 versus 23.8 p<0.0001). These findings indicate that chronic hypoxia reduces body weight and that suppression of mitochondrial complex II alleviates weight loss in chronic hypoxia. Body weight loss predicts increased mortality in chronic heart failure (Rossignol et al. 2015, European journal of heart failure, 17(4):424-433) and chronic obstructive pulmonary diseases (Wilson et al. 1989, Am Rev Respir Dis. 1989, 139(6):1435-8). Thus inhibiting complex II may be therapeutically relevant to prevent weight loss in such chronic heart and lung diseases.

Discussion

The present disclosure shows that pharmacologic inhibition of mitochondrial respiration in normoxia induces A3A-mediated RNA editing and the hypoxic transcriptome in primary monocytes. AtA5 and MXT reduce hypoxic gene expression in THP-1 monocytic leukemia and 293T embryonic kidney cell lines by antagonizing the stabilization of HIF-1α. Partial inactivation of MTCII by heterozygous gene knockouts of Sdh subunits blunts hypoxia-induced increases in hemoglobin levels in mice. Thus, inhibition of mitochondrial respiration activates the hypoxia responses in monocytes via a distinct mechanism.

These findings support a novel oxygen sensing and signaling mechanism for hypoxic transcript induction that is triggered by the inhibition of mitochondrial respiration in a cell type specific manner. To our knowledge, primary human monocytes are the first experimental model for SDH-mutated paragangliomas in mammals in which mitochondrial respiratory inhibition triggers transcriptome-scale responses to hypoxia. It is conceivable that other specialized cell types which depend on highly-oxygenated in vivo environments (e.g. arterial blood, alveolus) may utilize mitochondria, rather than the PHD-HIF system, for oxygen sensing to regulate hypoxic gene expression. Interestingly, mitochondrial inhibitors suppress rather than induce hypoxic gene expression in THP-1 monocytic leukemic cells suggesting that the hypoxia sensing apparatus switched from mitochondria to the PHD-HIF system in the THP-1 cell line. Based on our data, we consider that prolonged survival under chronic hypoxia in Sdh mice is caused by (1) enhanced hypoxia adaptation of some cell types such as monocytes by MTCII mutations (2) suppression of HIFs, whose prolonged activation is detrimental to survival as shown in animal models and human evolutionary studies on altitude-adapted populations. (3) reduced activity of TCA cycle, since Sdh is part of TCA cycle.

Materials and Methods

Cells, Cell Lines and Tissue Culture

Leukoreduction filters (Terumo BCT, Lakewood, Colo.), waste products of platelet donation process, were used to isolate PBMCs by an IRB-approved protocol. PBMCs were isolated using Histopaque-1077 (Sigma). Monocyte-enriched PBMCs (MEPs) were prepared using cold-aggregation method with slight modifications (30,60) Monocytes were isolated from MEPs or PBMCs using EasySep Human CD14 Positive Selection Kit (STEMCELL Technologies). Flow cytometric verification of isolated CD14+ cells were performed using RPCI core facility services. The MEPs were cultured at an average density of 25-35×10⁶/ml in 1 or 2 ml per well in 6- or 12-well standard tissue culture plates under standard conditions (37° C./5% CO₂) in RPMI-1640 medium with 10% FBS, 100 U/ml penicillin and 100 mg/ml streptomycin (Mediatech). Isolated CD14+ and CD14− cells were cultured at approximately 5×10⁶ cell/ml and 7×10⁶ cells per ml densities, respectively. THP-1 and TLA-HEK293T cell lines were purchased from ATCC, and Open Biosystems®, respectively, and cultured in recommended conditions. THP-1 cells were cultured in 10⁶ cells per 100 μl in 96-well culture plates in ATCC-formulated-1640 medium (30-2001), whereas 293T cells were cultured in DMEM medium supplemented with 10% FBS.

Hypoxia, IFN-1 and Inhibitors Treatment

Cells were cultured under 1% or 6% O₂, 5% CO₂ and 94% N₂ at 37° C. using Xvivo System (Biospherix). Human ‘universal’ type I IFN was obtained from PBL Assay Science and used at 600 U/ml. Atpenin A5 (Cayman chemical #11898), myxothiazol (Sigma-Aldrich, #T5580) and 2-Thenoyltrifluoroacetone (TTFA) (Sigma-Aldrich, #T27006) were used at 1-2 μM and 400 μM final concentrations, respectively. DFO (Sigma-Aldrich #D9533) and DMOG (Sigma-Aldrich #D3695) were used in 0.5 mM and 1.0 mM final concentrations, respectively.

Transfection of Plasmid DNA

HEK293T cells were cotransfected with the 400 ng of HRE-luciferase (Addgene, plasmid #26731), 1 ng of pRL-SV40 plasmid, 600 ng of pcDNA 3.1(+) (control empty vector) per well at ˜50-60% confluency using jetPRIME (Polyplus-transfection) in 12-well culture plates as per the manufacturer's instructions. Transfection efficiency was 60%-80% as assessed by fluorescent microscopy of cells co-transfected with the pLemiR plasmid DNA (Open Biosystems) for expression of a red fluorescent protein. Cells were harvested 2 days after transfection for measurement of their HRE and Renilla luciferase activities using Dual-Luciferase Reporter Assay System (Promega). HRE expression was quantified as a ratio of HRE/Renilla luciferase activities.

Immunoblotting of Cell Lysates

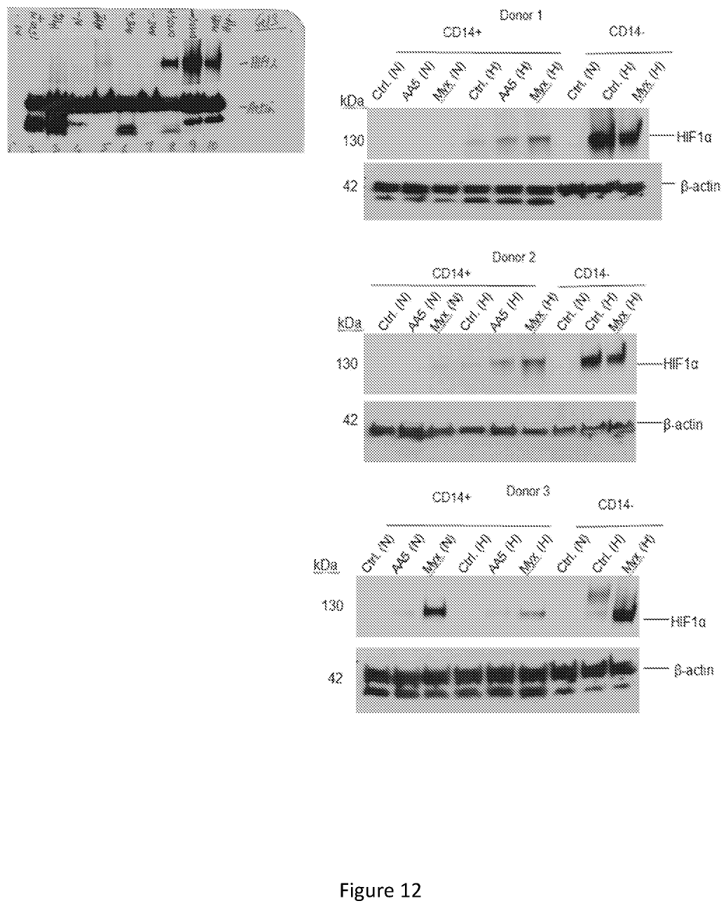

2× Laemmeli buffer (BIO-RAD) was used to prepare whole cell lysates. The lysate resuspended in the Laemmeli buffer was heated at 95° C. for 15 minutes, and 40 μl of the sample was used to perform gel electrophoresis on pre-cast, 4%-15% gradient polyacrylamide gels (Mini-PROTEAN TGX, Bio-Rad) in Laemmeli buffer system. Mouse monoclonal anti-HIF1α (product number GTX628480, GT10211; 1:1000 dilution) and mouse monoclonal anti-β-actin (product number AM4302, AC-15; 1:15,000 dilution) was used to detect HIF-1α or actin, respectively followed by incubation with HRP-conjugated goat anti-mouse antibodies (Life Technologies) at 1:2000 dilution. Bigger gel images of western blots of primary cells in FIGS. 1D and 4B are shown in FIG. 12.

Oxygen Consumption and L-Lactate Measurement

Oxygen consumption was measured using phosphorescent oxygen probe, MitoXpress-Xtra (Cayman Dual Assay Kit, item no. 601060). Monocytes were enriched to >50% purity by short-term cold aggregation and first cultured in standard conditions for 24 hours without treatment to stimulate metabolic activity. Cells were then centrifuged at 200×g for 7 minutes and resuspended in 1 ml RPMI/1% FBS with or without mitochondrial inhibitors. Cells are covered by mineral oil after addition of MitoXpress-Xtra following manufacturer's protocol. The fluorescence was kinetically measured on a plate reader (Synergy H1) at 20 sec intervals for approximately 3 hours (delay 70 μsec, collection time 30 μsec). Supernatants of the oxygen consumption assay were used to measure L-lactate levels following manufacturer's instructions.

Sdh Transgenic Mice and Hypoxia Exposure

Sdhb and Sdhc heterozygous mice in B6/129P2 background were gifts from Dr. Greg Cox (The Jackson Laboratory, Bar Harbor, Me.). The embryonic stem cell lines (Sdhb<6T(APO532)wtsi> and Sdhc<6T(BA0521)wtsi>) were generated by gene trapping (61) The gene trap vector insertion into Sdhb or Sdhc early introns creates fusion transcripts containing sequences from upstream gene exons joined to the β-geo marker, and interrupts the ORFs. Genetic verification of the knockout constructs was performed by genomic PCR and sequencing. A gene-specific intronic oligonucleotide PCR primer paired to either a vector-specific primer or another gene-specific intronic primer amplifies a knockout allele or a wild-type allele, respectively. We also re-derived a previously described Sdhd knockout mouse (Piruat et al., Mol. Cell. Biol., 24, 10933-10940) in C57BL/6J background at RPCI transgenic facilities using frozen sperm (mfd Diagnostics, Germany). Mouse genotyping was performed by tail DNA extraction using Allele-in-One Mouse Tail Direct PCR system (Allele Biotech) or by RPCI transgenic core facility.

Mice were exposed to chronic hypoxia (10%; range 9%-11%) using a vacuum operated hypobaric chamber (Case Western Reserve University Design Fabrication Center, Cleveland, Ohio). Oxygen percentage is continuously monitored by a sensor. The chamber accommodates two standard cages, each for five mice. Mice (several weeks after weaning) were initially subjected to approximately 17%-13% oxygen for six days and then chronically to 10% oxygen. The mice were exposed to room conditions for approximately 30 minutes each day during cage cleaning. Complete blood counts were obtained using automated cell counters Hemagen HC5 (cohorts A, B) or ProCyte Dx (cohort C) Hematology Analyzers. The mice were housed at RPCI core facility and studies were approved by IACUC.

RNA Seq and Bioinformatic Analysis

RNAs extracted from CD14+ cells were purified using RNA clean-up and concentration kit (Norgen Biotek corp.). Illumina TruSeq paired stranded total RNA with RiboMinus Gold kit was used to obtain sequencing libraries. Paired 101 bp RNA sequencing was performed on an Illumina HiSeq 2500 system (all nine samples in one flow lane). Raw reads passed quality filter from Illumina RTA were first pre-processed by using FASTQC(v0.10.1) for sequencing base quality control, then mapped to the latest human reference genome (GRCh38.p7) and GENCODE annotation database (version 25) using Tophat2(v2.0.13). Second round of QC using RSeQC(64) was applied to mapped bam files to identify potential RNA Seq library preparation problems. From the mapping results, the read counts for genes were obtained by HTSeq using intersection-strict option. Differentially expressed genes were identified using DESeq2, a variance-analysis package developed to infer the statically significant difference in RNA-seq data. Gene fold changes were calculated using regularized-log 2 transformation in DESeq2 R package. The raw RNA-seq data are submitted to the EMBL-EBI ENA archive under primary accession number PRJEB12121.

Other

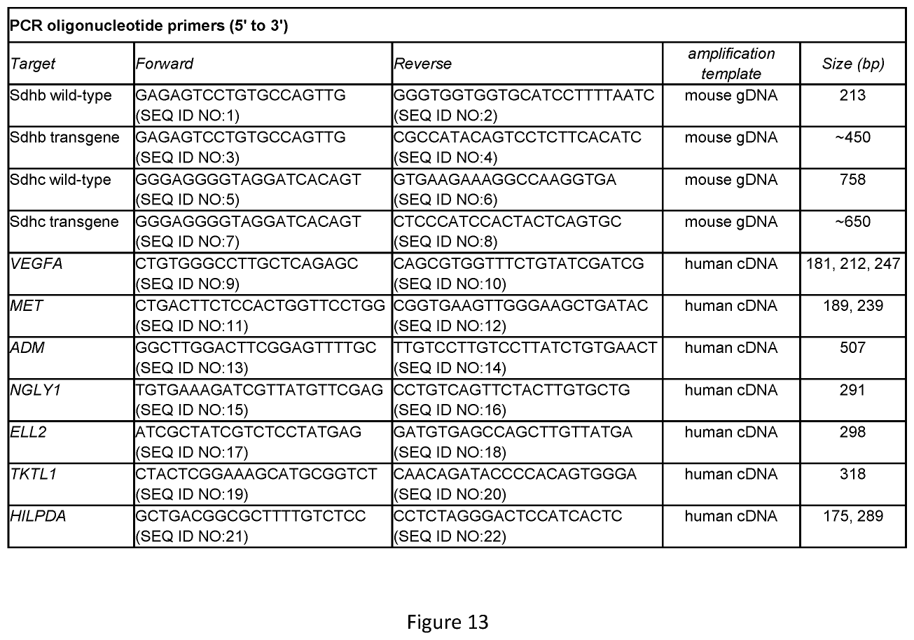

RNA and plasmid DNA were isolated with commercial kits (TRIzol, Life Technologies and Qiagen, respectively). RNA/DNA was quantified by Nanodrop 2000 (Thermo Fisher). Proteins were quantified using Bio-Rad Dc assay with BSA standards. RNA was reverse transcribed with the Transcriptor First Strand cDNA Synthesis (Roche) kit. SDHB c.136C>U RNA editing was quantified by allele specific RT-qPCR PCR oligonucleotide primers (FIG. 13) were obtained from Integrated DNA Technologies, Inc. ADM, ELL2, HILPDA, NGLY1, MET, TKTL1, VEGFA and B2M gene expression levels was assessed by qPCR using FastStart Taq DNA polymerase and SYBR Green I dye on a LightCycler 480 System (Roche). Quantification cycle values were calculated by the instrument software using the maximum second derivative method and the mean quantification cycle value of duplicate PCR reactions was used for analysis. Delta delta CT method is used to infer gene expression changes.

Statistical Analysis

Effects of inhibitors and hypoxia on RNA editing in biological replicates (PBMCs and MEPs) were initially tested by ANOVA, then by multiple comparisons (FIGS. 1 and 3C). Effect of inhibitors on VEGFA and HILPDA expression in biological replicates (FIGS. 4B, C) were tested by paired t-test (two-sided). Unpaired t-test (two-sided) was used to examine hemoglobin changes in mice and effect of inhibitors on HRE expression in HEK293T cells (FIG. 5D). False discovery rate approach was used to examine gene expression changes in THP-1 and HEK293T cells (FIG. 5). Statistical calculations were performed by GraphPad prism 7.00 software.

While the present invention has been described through various specific embodiments, routine modification to these embodiments will be apparent to those skilled in the art, which modifications are intended to be included within the scope of this disclosure.

What is claimed is:

1. A method of treating a systemic chronic low oxygen condition in an individual comprising administering to an individual in need of treatment a composition comprising a therapeutically effective amount of a mitochondrial complex II (MTCII) inhibitor.

2. The method of claim 1, wherein the MTCII inhibitor is Atpenin A5.

3. The method of claim 1, wherein the systemic chronic low oxygen condition is associated with COPD, chronic mountain sickness, cyanotic heart diseases, cystic fibrosis, obesity, obstructive sleep apnea, congestive heart failure, pulmonary embolism, asthma, idiopathic pulmonary fibrosis or acute respiratory distress syndrome.

4. The method of claim 1, wherein the Atpenin A5 is administered at a dose and frequency such that inhibition of mitochondrial complex II is maintained during the period of treatment.

5. The method of claim 4, wherein the Atpenin A5 is administered at a dose of about 0.05 mg/kg to about 5.0 mg/kg body weight.

6. The method of claim 5, wherein the Atpenin A5 is administered at a dose of about 0.5 mg/kg to about 5.0 mg/kg body weight.

7. The method of claim 1, wherein administration of the MTCII inhibitor results in reducing hemoglobin levels and/or reducing red cell distribution width.

8. The method of claim 1 further comprising measuring arterial blood oxygen tension prior to administration of the MTCII inhibitor, during treatment with the MTCII inhibitor, and/or after termination of the treatment with the MTCII inhibitor.

9. The method of claim 1, wherein the hypoxia is mild, medium or severe.

10. The method of claim 1, further comprising administration of supplemental oxygen to the individual.


SEQUENCE LISTING (22 sequences)

SEQ ID NO: 1 - DNA, 19 bp, Artificial Sequence
  Primer
gagagtcctgtgccagttg

SEQ ID NO: 2 - DNA, 24 bp, Artificial Sequence
  Primer
gggtggtggtgcatccttttaatc

SEQ ID NO: 3 - DNA, 19 bp, Artificial Sequence
  Primer
gagagtcctgtgccagttg

SEQ ID NO: 4 - DNA, 23 bp, Artificial Sequence
  Primer
cgccatacagtcctcttcacatc

SEQ ID NO: 5 - DNA, 20 bp, Artificial Sequence
  Primer
gggaggggtaggatcacagt

SEQ ID NO: 6 - DNA, 20 bp, Artificial Sequence
  Primer
gtgaagaaaggccaaggtga

SEQ ID NO: 7 - DNA, 20 bp, Artificial Sequence
  Primer
gggaggggtaggatcacagt

SEQ ID NO: 8 - DNA, 21 bp, Artificial Sequence
  Primer
ctcccatccactactcagtgc

SEQ ID NO: 9 - DNA, 20 bp, Artificial Sequence
  Primer
ctgtgggccttgctcagagc

SEQ ID NO: 10 - DNA, 23 bp, Artificial Sequence
  Primer
cagcgtggtttctgtatcgatcg

SEQ ID NO: 11 - DNA, 23 bp, Artificial Sequence
  Primer
ctgacttctccactggttcctgg

SEQ ID NO: 12 - DNA, 23 bp, Artificial Sequence
  Primer
cggtgaagttgggaagctgatac

SEQ ID NO: 13 - DNA, 22 bp, Artificial Sequence
  Primer
ggcttggacttcggagttttgc

SEQ ID NO: 14 - DNA, 25 bp, Artificial Sequence
  Primer
ttgtccttgtccttatctgtgaact

SEQ ID NO: 15 - DNA, 23 bp, Artificial Sequence
  Primer
tgtgaaagatcgttatgttcgag

SEQ ID NO: 16 - DNA, 22 bp, Artificial Sequence
  Primer
cctgtcagttctacttgtgctg

SEQ ID NO: 17 - DNA, 21 bp, Artificial Sequence
  Primer
atcgctatcgtctcctatgag

SEQ ID NO: 18 - DNA, 22 bp, Artificial Sequence
  Primer
gatgtgagccagcttgttatga

SEQ ID NO: 19 - DNA, 22 bp, Artificial Sequence
  Primer
ctactcggaaagcatgcggtct

SEQ ID NO: 20 - DNA, 22 bp, Artificial Sequence
  Primer
caacagataccccacagtggga

SEQ ID NO: 21 - DNA, 21 bp, Artificial Sequence
  Primer
gctgacggcgcttttgtctcc

SEQ ID NO: 22 - DNA, 21 bp, Artificial Sequence
  Primer
cctctagggactccatcactc